(19) United States
(12) Patent Application Publication
(10) Pub. No.: US 2009/0215027 A1
(43) Pub. Date: Aug. 27, 2009

(54) Method and system for colorimetric determination of a chemical or physical property of a turbid medium

(75) Inventors: Ulf Houlberg, Lynge (DK); Peer Herbsleb, Stovring (DK); Joseph Sturino, College Station, TX (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Chr. Hansen A/S, Horsholm (DK)

(21) Appl. No.: 10/586,325

(22) PCT Filed: Jan. 17, 2005
(86) PCT No.: PCT/DK05/00027

§ 371 Date: Sep. 5, 2007

Related U.S. Application Data
(60) Provisional application No. 60/536,832, filed on Jan. 16, 2004.

(51) Int. Cl. C12Q 1/70; C12Q 1/04; C12M 1/34

(52) U.S. Cl. 435/5; 435/34; 435/288.7

(57) ABSTRACT

A new method and a system for the simultaneous determination of a biological, chemical and/or physical property of a plurality of individual samples of a turbid is described. The invention relates to a system and colorimetric method for simultaneous determination and measuring properties, such as acidification or pH value, redox potentials, viscosity, diffusion, enzymatic activity, etc. of a plurality of individual samples of a turbid or opaque medium, such as, e.g. milk, whey and related products. In particular, this invention relates to a method for non-invasively and/or non-destructively scanning samples or an array of samples, and determine on the basis of the scanning a specific property, such as pH, of the samples. The method may also be used for multivariate determinations of chemical and/or physical properties.

### FIELD OF THE INVENTION

This invention relates to a method and a system where a chemical and/or physical property or characteristic of a turbid medium can be determined by a quantitative translation of a digital image of its color. In particular, this invention relates to a system and calorimetric method for simultaneous determination and measuring properties, such as acidification or pH value, redox potentials, viscosity, diffusion, enzymatic activity, etc. of a plurality of individual samples of a turbid or opaque medium, such as, e.g. milk, whey and related products, where said method may be automated to obtain data from a large number of samples over an extended time period. In particular, this invention relates to a method for non-invasively and/or non-destructively scanning samples or an array of samples, and determine on the basis of the scanning a specific property, such as pH, of the samples. The method may also be used for multivariate determinations of chemical and/or physical properties.

### BACKGROUND OF THE INVENTION

Within the food industry it is customary to monitor several chemical and physical properties of food products in order to ensure a standardized product. Dairy products are characterized by being generally turbid or non-transparent, and fermented milk, such as yogurt, often has a specific acidity and a desired viscosity. The resulting whey from cheese making contains lactic acid bacteria that have been added to the cheese milk to provide a desired fermentation and flavour characteristics. However, bacteriophage infection of the lactic acid bacterial cultures used in the fermentation may result in less lactic acid production by the bacteria and poor yield. Thus, culturing of lactic acid bacteria from whey and analysis of the cultures for the monitoring of bacteriophages are routinely done. Many resources are used in the dairy industry to monitor pH in milk in a range of different applications. Most of these applications are in volumes of 100 ml or 200 ml. These volumes make it difficult to automate the procedures and the screening work is a heavy burden.

It is desired to provide a simple method for measuring acidity and/or viscosity of milk related products, such as whey where less than desired acidification can be correllated to detection of bacteriophages, and yogurt where both a desired pH level and a desired viscosity are important. It is preferred that the method can be subjected to automation and handling of a large number of samples, and that the samples used can be contained in microtitre plates.

In standard forms of pH measurements of a sample of a milk related product, the use of electrodes in physical contact with the sample is generally required. This has for instance been described in the Japanese patent application no. JP 97274007, in which a color system is disclosed for displaying pH levels of a fluid. The system comprises a counter electrode and a reference electrode inserted in a sample placed in contact with a surface of the substrate and a resistance electrode, which is connected to the substrate. The system establishes a DC voltage between the resistance electrode and the counter or reference electrode. The DC voltage generates a depletion layer in the substrate, in which layer a photocurrent flows in accordance with light emitted by a laser. The photocurrent reflects the pH level of the sample in contact with the surface.

Persons skilled in the art normally consider the use of probes, such as electrodes, that are inserted into a sample as a complication in terms of measurement efficiency since the probes continuously need to be cleaned and calibrated. An additional complication related to pH measurement is that the electrodes tend to drift during an analytical operation, which thereby easily generates an incorrect measurement of pH. As a further complication, the use of probes per se significantly reduces the speed at which a determination of the desired property of a series of samples may be obtained, as the probes are to be moved from one sample to the next.

As an example of the use of chromatic indicator material for pH measurements of a sample, the following applies: A light-emitting element such as a light emitting diode in conjunction with a chromatic pH sensitive material as described in the international patent application WO 01/94921 in which a pH sensor system is disclosed. The system is capable of measuring the pH level of a sample based on the characteristics of a chromatic pH sensitive material used in the system. The system utilises a light emitting diode (LED) for providing light communicated to a chromatic material layer added to one surface of a transparent container. When the ambient pH level of the sample reaches a predetermined level the chromatic material saturates and a light sensitive circuitry can measure a difference in the intensity of the light emitted by the light emitting diode. The chromatic material provides an indication when the pH level of the sample has reached a specific level. However, the system does not enable measurement of specific pH values. Therefore, although this system may be used for determining whether a product is usable or non-usable it does not provide a more detailed outline of the pH level, which in many cases is required.

A further example of using chromatic indicator material for bioactivity determinations is described in European patent application EP 0301699. A method and an apparatus for determining the bioactivity of liquid biological mixtures are described. The method comprises directing visual spectrum light to a liquid biological sample. Subsequently, the reflectance of the light is detected by the use of tristimulus readings for one sample at a time and e.g. correlated to a pH standard.

Standard rheometry measurements (measurement of flow of viscous substances) involve relatively large samples of the medium to be tested. Accurate measurements of viscosity parameters are performed by different viscometry methods where shear rates are applied to the measured product under highly controlled conditions (dimension of measuring systems, speed, temperatures, etc.) and the resulting stress parameters are recorded. These types of measurement are accurate and reproducible but highly demanding in regard to required time per sample, technical skills and precision. More empirical measures of viscosity can be obtained from the outlet time for a standard volume to pass through a Posthumus funnel or a Brookfield viscometer (Brookfield Engineering Laboratories, Inc., Massachusetts) with different measurement systems (spindles, bob-cup, etc.) and with or without Helipath stand. Typical sample volumes necessary for rheometry are from about 1 ml up to 1.5 L. Also these types of measurements are demanding in regard to required time per sample and none of them are suited for screening purposes of large series of samples.

Thus, until now accurate, fast, cheap and easy methods and systems for simultaneous determination of chemical or physiological properties of a plurality of individual samples have not been provided.

### SUMMARY OF THE INVENTION

An object of the present invention is to provide a method and system utilising determination of a sample of a turbid medium and correlate the captured signal with a specific chemical or physical property of the medium. In particular, it is an object of the present invention to provide a method and system for determining a graduated image, such as an image of a chemical or physical property of a sample with high accuracy and speed and without requiring probes to be inserted into the sample. In essence the present invention provides a method and system for simultaneous, nearly continuous and non-invasive determination of a chemical or physical property of a plurality of individual samples. Said purpose being obtained by the following method of the invention:

A method for simultaneous determination of a chemical and/or physical property of a plurality of individual samples of a turbid medium comprising the steps:
i) arranging said samples of the medium comprising a color indicator in an array;
ii) allowing said color indicator to interact with said samples;
iii) providing picture capturing means for capturing a digital image of the color developed on a surface of said samples following said interaction;
iv) using said digital image to obtain a digital value representation for said property said value representation being used for calculating a value for said property.

### BRIEF DESCRIPTION OF THE DRAWINGS

The above, as well as additional objects, features and advantages of the present invention, will be better understood through the following illustrative and non-limiting detailed description of preferred embodiments of the present invention, with reference to the appended drawings, wherein:

FIG. 1 illustrate a flow chart of a method according to one embodiment of the present invention.

FIG. 2 illustrate a system set-up according to one embodiment of the present invention.

FIG. 3 shows a flow chart of a computer program according to a preferred embodiment of the present invention.

FIG. 4 shows the standard curve, pH vs. Hue

FIG. 5 shows a standard curve of transformed (linearised) Hue value (f(Hue°)) and pH.

FIG. 6 shows Acidification curves for eight different concentrations of Lactococcus lactis O-culture (R 604). The right hand legend indicate inoculation level from 0.002% to 3.333%. the pH values are converted from transformed values as described in Example 2.

FIG. 7 is a graph showing measured pH vs. electrode measured pH. Measurements of pH standards and samples are shown on the left hand axis and the calculated difference between the standard and the sample measurements is shown on the right hand axis.

FIG. 8 shows a flow diagram and procedural elements (or steps) of a current method, the spot test.

FIG. 9 shows a flow diagram and procedural elements (or steps) of the colorimetric microassay of the invention.

FIG. 10 shows pH data from an example of an assay for the determination of phage infection in of a mesophilic lactic acid bacteria (a host array) as described in Example 3. The pH values are the values showed in circles. The circles are illustrating wells in a multiwell or microtiter plate. Position A1 to B12 are duplicate pH standards, used to establish the pH determination of the assay. Position C1 contained, Indicator Milk+10⁸ PFU/ml phage and no indicator bacteria; C2, Indicator Milk+10⁸ PFU/ml phage+indicator bacteria; C3, Indicator Milk+10⁶ PFU/ml phage+indicator bacteria; C4, Indicator Milk+10⁴ PFU/ml phage+indicator bacteria; C5, Indicator Milk+10² PFU/ml phage+indicator bacteria; C6, Indicator Milk and indicator bacteria only. Position D1 to D6 is a duplicate of C1 to C6. The situation at 0 and 4 hours of incubation is shown.

FIG. 11. Time for diffusion of indicator through milk fermented with 5 different cultures. The horizontal bars indicate standard deviations to the mean value for tree independent acidifications. A high Tdiff value (Y-axis) indicate a high viscosity/texture. “Low, middle and High” above each culture shows the known texturing capabilities of the yogurt cultures.

FIG. 12 Precision of the pH measurements in milk. The same sample of reconstituted skim milk added 2.5 ml of each indicator, Bromcresol purple and Bromcresol green, was repeatedly scanned every 2 min. Pictures were captured and analysed using the pH-Scan 2.0.00 program, and the calculated pH values shown as a function of time.

FIG. 13. shows part of a flatbed scanned picture of the microtitre plate. Column 1 and 3 contained the pH standards. Column 3, Position D-H are empty wells. Column 2 contains samples with a decreasing concentration of culture (concentration of culture increasing from left to right). The inoculation level is stated in the line “Inoculation”, inoculation level is given in % vol/vol.

FIG. 14. Viscosity/texture of Streptococcus thermophilus strains grown in milk determined by the “color of viscosity” method (X-axis) vs. measurement in a rheometer (Y-axis). A high value for Tdiff and in the rheometer corresponds to a high viscosity.

### DETAILED DESCRIPTION OF THE INVENTION

More specifically the method of the invention further comprises the step of comparing said digital value with the values obtained from a standardised set of samples having a range of known values representing said property to obtain a calculated value for said property.

In principle, there are no limitation as to the plurality of samples that can be simultaneously subjected to the method of the present invention. However, in presently convenient embodiments said plurality of samples may be arranged in the form of an array of samples or they may be in the form of a container such as a microtitre plate (multi well plate) having a plurality of wells, said plurality being in the range of between 2 and 4000, such as 6, 24, 96, 384, or 1536 wells. 96, 384 or 1536 wells are preferred. 96 well plates typically require a working volume including sample per well of 100 to 200 μl, 384 well plates typically have a working volume per well of about 2-20 μl, and the Nunc™ 1536 well plates have a total volume of 13 μl per well allowing the use of very small samples of about 1 g. Thus, a large number of samples combined with low single sample volume can be obtained using the method of the invention.

Preferably, the container has at least one transparent surface, such as a top or a bottom, through which the color development of the sample may be determined.

It is preferred that said picture capturing means for determination of color is a color-enabled photoelectric scanning device, optionally with a color measuring head being movable under computer control, that produces a digital color representation of said surface; said means being preferably a scanning device, such as a line-by-line operating autofeed scanner, a flatbed scanner, a digital camera, a high speed digital camera and a high speed video imaging wherein said picture capturing device preferably operates on an at least partly open or transparent end of said array or container, e.g. through the bottom of said container, to generate an image file recording of the color of said samples.

The use of a high speed digital camera or even high speed video camera as picture capture means will allow pictures of the array of samples to be captured with very short intervals. A number of suitable cameras are commercially available. One example is the Phantom v 7.1 camera, Vision Research Inc., USA (www.visiblesolutions.com). This camera offers a recording speed of 4800 full frame pictures per second, thus enabling a user of the system or method of the present invention non-invasive determination of a chemical or physical property of a plurality of individual samples determined at infinitesimal small time intervals resulting in nearly continuous determination useful for determinations of e.g. growth kinetics, enzyme kinetics, chemical reactions and other continuous processes which can be visualised by means of a color indicator.

Digital image processing methods may be used to obtain an image file for determining the measuring positions from the digital color representation of said surface and for calculating said value for said property.

A specific embodiment of the method of the invention comprises illuminating the at least partly transparent surface of the samples and/or container in connection with determining said color, and, optionally, the further steps of
i) analyzing said image file and generation of data values for image parameters by means of an analyzer; and
ii) translating said data values for image parameters to a value representing said chemical and/or physical property of said sample by means of said analyzer.

### Turbid Media Useful in the Method of the Invention

The method of the invention is adapted to measure the reflected color of a surface of a plurality of individual samples of the turbid or non-transparent medium to be analysed. Thus, it is Important to avoid interfering reflections from the interior of the sample and/or from the walls of the container holding the sample.

Turbidity is a measure of the cloudiness of fluids, such as waste water and other aqueous fluids, which is a function of the amount of suspended and dissolved material. Turbidity can be defined as a decrease in the transparency of a solution due to the presence of suspended and dissolved substances, which causes incident light to be scattered, reflected, and attenuated; the higher the intensity of the scattered or attenuated light, the higher the value of turbidity. There are various methods for measuring turbidity of solutions and colloidal liquids. However, a standardized method that can be used for all liquid media and for the purposes of the present invention does not exist. A preferred or useful lower limit of turbidity of the medium to be analysed in the method and system of the invention will also depend on the intrinsic of the medium and it is advised that the skilled worker establishes a useful lower limit of turbidity for each medium to be analyzed.

A preferred medium has a light color or is white before addition of the indicator. However, it is conceivable that a strongly reflecting medium, such as whole blood, could be used in the method of the invention where the property to be measured can be visualized through the use of an indicator that operates on the sample by bleaching of the hemoglobin or by shifting the reflectance of the blood, thus enabling a specific property of the blood, such as an enzymatic activity or the concentration of a blood gas, to be quantified.

The medium is preferably in the form of a liquid, a semi-liquid or a gel. The medium is characterised in being turbid, which for the purposes herein shall mean that it appears non-transparent or opaque due to its cloudiness.

The medium is preferably selected from the group consisting of biological fluids, such as dairy products, oil products, fruit juice products including jelly, spice products, beverages, whole blood, or any combination thereof; as well as emulsions including mayonnaise, salad dressings; cosmetic products, such as skin lotions, skin tonics; paints, soaps and other technical emulsions. The medium may contain live microorganisms, such as yeasts or lactic acid bacteria or both, an example being various fermented milk products and whey, unfiltered beer and other opaque fermented beverages. The medium may be a bacterial suspension, or the medium may be a mixture of any of the above. The medium may further comprise solids, such as suspensions and dispersions. Any solid material present in the medium should be comminuted or in the form of particles, and the medium should appear homogeneous and of uniform.

Chemical and physical properties of the medium to be measured by the method and system of the invention include acidity, viscosity, gel strength, enzymatic activity, such as peroxidase activity.

### Indicators Useful in the Method of the Invention

An indicator useful in the present method is characterized in being able to impart a change as a function of the property of the medium to be measured and includes any indicator dye.
A typical example is a pH indicator that changes according to a specific pH level of the medium. Useful indicators for measuring pH in specific ranges of about 1 to about 2 units in the range from pH=0 to pH=13 include crystal violet, cresol red, thymol red, erythrosin B, 2,4-dinitrophenol, bromphenol blue, methyl orange, bromcresol green, methyl red, Eriochrome™ Black T, bromcresol purple, alizarin, bromthymol blue, phenol red, m-nitrophenol, o-cresolphthalein, phenolphthalein, thymolphthalein and alizarin yellow GG. One or more pH indicators can be used in the method of the invention depending on the pH range to be measured. The combination of bromcresol green and bromcresol purple will provide an adequate signal in the range from about pH=4 to about pH=7 which is useful for most dairy products.
Another example is a indicator, such as the dye brilliant blue which does not react with the medium, but will penetrate the medium evenly as a function of the viscosity of the medium. Such dyes may also be used to measure diffusion rates in a medium.
Oxidation-reduction indicators (redox indicators) are mostly brightly colored when oxidised and less reduced, e.g. 2,6-dichlorophenol indophenol. They can be used to detect the redox potential of a particular solution or may be used as electron donors or acceptors in which case the rate of a redox reaction can be followed. The rate of reduction of dye can also be used as an enzyme assay, e.g. for succinate dehydrogenase. As reduction takes place the color of the dye changes. The electron transport processes of chloroplasts, mitochondria, bacteria, yeasts, homogenates and even tissue slices can be studied using indicators. Examples are tetrazolium salts which upon reduction yield an insoluble brightly red ed compound (formazan). Methyl viologen and benzyl viologen (less when oxidized and brightly ed when reduced) are used to demonstrate the presence of a strongly reducing system. They can be reduced by photosystem 1 of chloroplasts but not photosystem 2.

Useful indicators for determination of viscosity (rheometry) and/or diffusion rates include, e.g., brilliant blue and the like for media such as yogurt.

### Digital Recording and Data Processing

A particular feature of the present invention relates to the provision of a digital recording of the measurement enabling a user of the system or method to continuously monitor the measurements and recall specific recordings for comparisons.

A specific aspect of the present invention is a method for determining a pH value of a plurality of individual samples comprising:

(a) adding a pH indicator indicating a specific pH level with a specific color to a medium
(b) arranging said medium in an array;
(c) incubation of said samples in said array and scanning said array by means of a scanner or digital camera thereby generating an image file recording color of said pH indicator;
(d) analysis of said image file and generating data values for image parameters by means of an analyzer; and
(e) translation of image parameters to a pH value of said individual samples by means of said analyzer used in step (d).

For use in determining the pH of dairy products and other foodstuffs the indicator according to the present invention may be adapted to indicate pH levels between 4 and 8. Alternatively, the pH indicator may be adapted to indicate smaller ranges of pH levels such as pH values between 3 and 4, 4 and 5, 5 and 6, 6 and 7, or 7 and 8. Obviously, the pH range size may be adapted to the possible wide variety of types of samples to be measured in accordance with the requirements to accuracy and precision.

Utilisation of the method according to the invention ensures that measurements may be performed continuously and efficiently since the samples may be exchanged for other samples without undue burden.

In addition, the method of the present invention when measuring properties, such as pH or viscosity, is inexpensive to perform since expensive electrodes for measuring e.g. a pH level are avoided. Further, connecting cables between the electrodes and an analyzing means such as a pH meter is completely avoided.

The method according to the invention may be used in the dairy industry for screening/determining of the acidification activity of the various strains of lactic acid bacteria cultures including reduced or lack of acidification activity due to bacteriophage infection, and screening of formulation mixtures or substrates in connection with for example lactic acid bacteria. In fact, the method may be used in a wide variety of processes involving property changes, such as pH changes, in any size volumes. Particularly, the method is advantageous for automatically measurement of pH in samples contained in microtitre plates.

The method of the present invention may further comprise adding bacterial cultures to the samples in the container. The adding of bacterial cultures enables one to determine the acidification activity of bacterial cultures. For example, screening of formulation mixtures or substrates with lactic acid bacteria.

The image file according to a first aspect of the present invention may comprise an image format such as: Synchronized Multimedia Integration Language (SMIL) format, any JPEG format, any Graphics Interchange Format (GIF), Computer Graphics Metafile, TIFF, BIFF, bmp, Clear, FITS, NFF, OFF, PCX, PNG, TGA, XBM, mod, Portable Document Format (PDF), Portable Network Graphics, Portable Pixmap, progressive coding, Quicktime, RIFF, Self Extracting Archive, sequential coding, server-parsed HTML, sprite, Tagged Image File Format, targa, Targa Graphics Adaptor, thumbnail, wav, WebCGM, wireless bitmap, xpm or a different frame rate video or similar format. The listed formats each provide benefits for specific applications and thus the image file in either of these formats provides a great flexibility and compatibility with any customer hard- or software.

The image parameters according to the first aspect of the present invention may comprise parameters known in the art for expression of color e.g. lightness, chroma, Hue, saturation, angle, or any combination thereof. The skilled person will immediately appreciate the meaning of these parameters and will further appreciate that any specific color expression system may be used in accordance with the present invention. One example of a colour system is the L*a*b* color space (also referred to as CIELAB). This color expression system is presently one of the most popular color space for measuring object color and is widely used in virtually all fields. Obviously, the present method is not depending on any particular color expression system and therefore any present or future color expression system will also apply in the method of the present invention. An extensive description of the various color representation parameters can be found in the pamphlet: Minolta: Precise Color Communication, “color control from feeling to instrumentation” Minolta CO., Ltd, 1994, which is hereby incorporated by reference.

The image parameters provide a wide variety of possible ways to determine the desired property, such as the pH level of the sample. The Hue angle determined from said image file correlates with a pH level of the sample. It is an important feature of the invention that by using the Hue angle for translation to the pH level the method becomes relatively insensitive to pH indicator concentrations in the container.

The scanning according to the first aspect of the present invention may comprise scanning of an at least partly transparent surface of the container. Alternatively and/or additionally the scanning may comprise scanning of an at least partly open end of the container. That is, the scanning may be performed on a surface of the container, which is at least partly transparent. Similarly, the scanning may be performed on one end of the container, which end may comprise one or more openings, so as to provide the optimal recordation of colors.

The term partly transparent should in this context be construed as one or more areas of the container being transparent or open and/or as one or more areas of the container being partly transparent.

The scanning of the container may comprise scanning the container in predetermined time intervals in the range of from about 0.00001 second to about 60 to 120 minutes. The container may thus be scanned with a series of scans so as to record time dependent changes of the chemical or physical property, such as the pH level of the sample. Obviously, the range could in fact be extended to days or months or be indefinite for continuous monitoring of said property, when keeping in mind handling of the data. That implies that the amounts of data to be recorded may require a certain size of memory and appropriate memory management.

The container according to the first aspect of the present invention may comprise a microtitre plate having a plurality of wells, said plurality being in the range between 2 and 4000, such as e.g. 6, 24, 96, 384, or 1536 wells. The use of a microtitre plate in the method of the invention enables the simultaneous measurement of specific arrays of samples. The microtitre plate may further comprise an at least partly transparent surface enabling the scanner to scan the samples contained in the wells. The scanner may perform the scanning of the container on the top surface comprising one or more openings for the wells or may perform the scanning of the container on another surface, such as the bottom surface, of the container being at least partly transparent. The at least partly transparent surface may comprise a transparent area positioned opposite to the open end of the well of the microtitre plate.

The analysis of the image file may be performed in predetermined regions of the container or array of individual samples. The analyzer performing the analyzing of the image file may be configured so as to analyze specific regions or areas of the container or array.

The method according to the invention may further comprise saving the image parameters in a data file. The data file may be a comma-separated-value type file, a space-separated-value type file, a text type file, or any combinations thereof. The data file may be configured in accordance with any software particulars so as to comply with a specific software standard or file requirement.

The method according to the invention may further comprise presenting the data file in graphical or textual form by means of a display. The display may comprise any type of monitor for presenting textual or graphical data such as a personal computer monitor, personal digital assistant monitor, and cellular phone display.

The analyzer according to the invention may comprise a processor such as in a computer, a server system, a personal digital assistant, a cell phone, or any combination thereof. The analyser may further comprise a memory device for storage of an analyzing program code to be executed by the processor, for storing image files recorded by the scanner, and for storing data values generated by the analyzer. The memory device may be connected to the processor through a computer network such as a dedicated line network, a local area network, a wide area network, a metropolitan area network, or an inter-network (e.g. the Internet). Similarly, the scanner may be connected to the processor through the computer network. Hence the scanner, the processor and the memory device may be separated so as to allow for the processor receiving data from a plurality of scanners located in various positions of for example a production line and so as to enable the processor to utilise a memory bank.

By utilising a computer for analysing a particularly advantageous and versatile solution is achieved as the computer may be used for any analytic functions run by any form of software packages.

Since the scanner performs the scanning of the sample a plurality of samples may be scanned simultaneously by including a plurality of containers in a scan or as described above by utilising a container, such as a microtitre plate, having a plurality of wells each containing a sample to be investigated. The operator's safety is enhanced by applying a scanner instead of utilising electrodes since the scanner removes the operator from the sample.

A further advantage of the method according to the invention is the possibility of performing a scan of samples through a surface which enables a scan of encapsulated samples. Thus, prohibiting any contamination of the samples as well as prohibiting an operator to become contaminated by the samples.

The above mentioned objects, advantages and features together with numerous other objects, advantages and features, which will become evident from the below detailed description, is obtained according to a further aspect of the present invention which is a system for determining a chemical or physical property, such as a pH level, of a plurality of samples and comprising the following elements:

A container for containing a plurality of individual samples;

A indicator to be introduced into said samples, which indicator is adapted to indicate a specific chemical or physical property with a specific color;

An incubator for supporting said container and incubating said samples contained in said container;

A scanner for scanning said container and thereby generating an image file recording color of said samples; and

An analyzer for analyzing said image file and generating data values for image parameters for said image file and determining said specific chemical or physical property of said samples from said image parameters.

The system according to the present invention provides means for determining a chemical and/or a physical property, such as obtaining a pH level, of the samples in the container. The system may be realized in a wide variety of technical ways such as a test kit thus providing for specific customer requirements to be incorporated. The system may establish a standardized way of measuring a property, such as a pH value, a viscosity value, a redox potential, an enzymatic activity of a sample. A chemical property such as the pH may further be correlated with a biological property in an otherwise well defined system. An example of this is the quantitative determination of acidification activity, i.e. the ability of a strain of lactic acid bacteria to acidify milk, cf. Example 1 and 2 below.

The system according to the invention may utilise any common elements obtainable on the market thereby presenting a fairly inexpensive system.

The above objects, advantage and feature together with numerous other objects, advantages and features, which will become evident from the below detailed description, is obtained according to a still further aspect of the present invention, which includes a computer program comprising a code that is adapted to perform the following actions when said program is run on a data processing system:

Control of picture capturing/scanning of an container containing a plurality of individual samples;
Generation of an image file of one surface of said container;
Identification of a color of said one surface of said container;
Analysis of said image file and generation of data values for image parameters; and
Translation of image parameters to a chemical and/or physical property of said samples.

The computer program useful in the present invention accomplishes the task of correlating the value for the specific chemical or physical property, such as a pH value, indicated by the color of the individual samples in the container and the image parameters determined from the scanned image file of the samples in the container.

The computer program may obviously be implemented as executed in one sequence or as a plurality of concurrently executed sequences. This possibility provides the opportunity of, e.g., applying the computer program in a production line or in a laboratory set-up.

The system and the computer program according to the invention may comprise any features of the method according to the invention.

In the following description of various embodiments, reference is made to the accompanying figures, which form a part hereof, and in which are shown by way of illustration various embodiments in which the invention may be practiced. It is to be understood that other embodiments may be utilized and structural and functional modifications may be made without departing from the scope of the present invention.

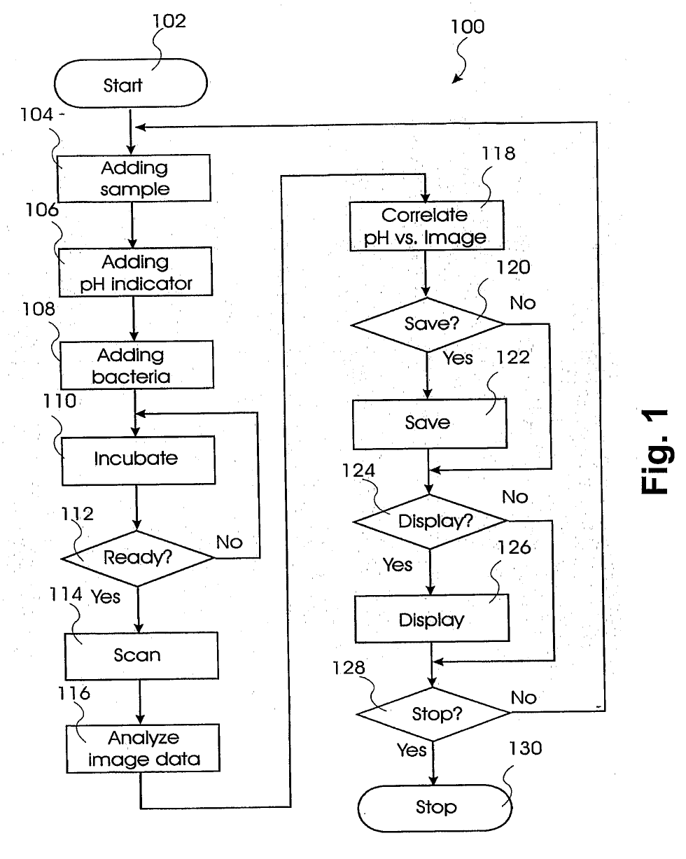

FIG. 1 shows a method designated in its entirety by reference numeral 100, which method 100 is one embodiment of the present invention.

The method 100 comprises a start step 102 during which the fundamental requirements such as data format and implementation procedure is established.

Following the initiation provided for by the start step 102, the method 100 enters an adding sample step 104 during which the method 100 adds a sample to be investigated in a container,

Subsequently or simultaneously the method 100 enters an adding indicator step 106 during which the method 100 adds a indicator to the container. In this example the indicator provides a color in accordance with a pH value in the sample. The pH indicator may respond to any pH value in the sample and may change color relative to small changes of pH value thus providing a high resolution.

The method further comprises an adding bacteria step 108 during which the method adds bacteria to the sample so as to determine the acidification activity of bacteria cultures. This step is particularly important in the dairy industry in connection with lactic acid bacteria screenings.

Following the introduction of the bacteria to the sample in the method 100 the contents of the container are incubated during incubation step 110 in a period determined by the Ready ? step 112. The time period during which the sample needs to be incubated may be determined during any of the preceding steps 102, 104, 106 and/or 108. The period may be determined in accordance with inter alia the characteristics of the sample, pH indicator and/or the bacteria, which are to be added to the container.

When the “ready” step 112 returns a “YES” the scan is initiated during scan step 114. The container is scanned on one or more surfaces, which is at least partly transparent or open so as to allow for the optimal scan to be performed. The scan is to generate an image file, which may comprise any data format known to a person skilled in the art. It is obvious that the data format may advantageously be configured in accordance with the requirements of a given analytic computer program.

Following the scan in the scan step 114 the method 100 proceeds to an analyzing step 116. During the analyzing step 116 the image file is investigated and image parameters determined. The analyzing step 116 may be operated according to any appropriate specifications, such as e.g. analysis of predetermined regions or areas of the container, and may determine predetermined image parameters, or any combination thereof. An example of an analysis predetermined area could be a microtitre plate containing a plurality of wells.

The image parameters may comprise lightness, chroma, Hue angle, or any combination thereof. The method 100 utilises the image parameters for determining the pH value of the sample during a translation step 118.

The translation step 118 provides data values for the pH level of the sample. Thus, in the example of the microtitre plate, the translation step 118 provides data values for pH levels of each of the wells in the microtitre plate. The data values generated during the translation step 118 may be saved.

The method 100 comprises a Save ? step 120 during which the method 100 determines whether the data values and/or the image file are to be saved on a memory device. Similarly, as described with reference to the Ready ? step 112, saving of the data values and/or the image file may be determined during any of the previous steps 102, 104, 106, 108, 110, 112, 114, 116, 118, or any combination thereof. The data values are saved during the save step 122.

The method 100 further comprises a Display ? step 124 during which the method 100 determines whether the data values and/or the image file are to be shown on a display. Similarly, as described with reference to the Ready ? step 112 and the Save ? step 120, displaying the data values and/or the image file may be determined during any of the previous steps 102, 104, 106, 108, 110, 112, 114, 116, 118, 120, 122, or any combination thereof. These data values may be conveniently be displayed during the Display step 126.

Following the Display step 126 or the bypass of the same the method 100 enters a Stop ? step 128 during which the method determines whether the method 100 should terminate and continue to a Stop step 130 or return to the addition of a Sample step 104. Similarly, as described with reference to the Ready ? step 112, the Save ? step 120, or the Display step 126 whether the method 100 is to return to the addition of a Sample step 104 or terminate in the Stop step 130 may be determined during any of the previous steps 102, 104, 106, 108, 110, 112, 114, 116, 118, 120, 122, 124, 126 or any combination thereof. Hence, the method 100 may comprise analysis of one sample in a first container or a series of analyses of a plurality of samples in a second container.

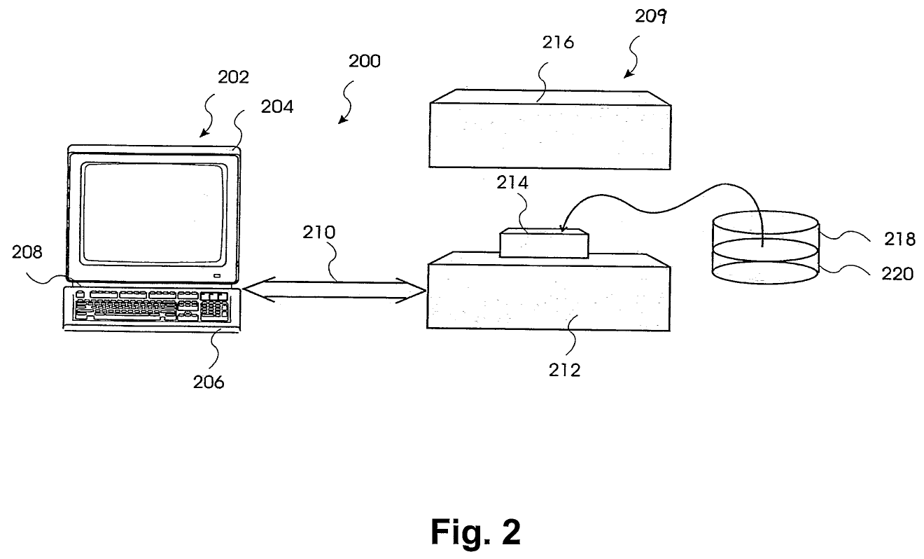

FIG. 2 shows a system according to a preferred embodiment of the present invention and designated in entirety by reference numeral 200. The system 200 comprises means of implementing and executing the method 100 described with reference to FIG. 1.

The system 200 comprises a computer 202 having a display unit 204, a keyboard 206, and a processor and memory unit 208. The processor may comprise any microprocessor or micro-controller known to a person skilled in the art, such as Intel® 80535, Intel® 8031, Intel® 80586 (Pentium), transputer processor, 64 bit processor, or any combination thereof. The memory may comprise electric, magnetic and/or optical recording medias.

The elements of the computer 202 are by way of example shown in FIG. 2 as being situated next to one another. However, a physical close location is not a requirement. Another example of location of the display unit, keyboard and the processor and memory unit may be to place them at separated locations linked through a communications network.

The computer 202 is connected to a measurement kit designated in entirety by reference numeral 209. The computer 202 is connected to the measurement kit 209 through connection 210. The connection 210 may be implemented as a dedicated line, a local area network (LAN), wide area network (WAN), a metropolitan area network (MAN), or an inter-network (the Internet). Hence the computer 202 may utilize the connection 210 for connecting to a plurality of measurement kits and executing a plurality of methods 100, as described with reference to FIG. 1.

The measurement kit 209 comprises a flat bed scanner 212 for scanning a surface of a container 214 containing one or more samples to be investigated and comprises an incubation unit 216 for incubating the contents of the container 214. The measurement kit 209 further comprises a indicator, such as a pH indicator, 218 in an indicator container 220 (e.g. one well in a microtiter plate), which indicator 218 is to be introduced in the container 220 prior to incubation of the sample. The scanner 212 is adapted to generate an image file of the surface of the container 214 and transfer the image file to the computer 202. The computer 202 is adapted to receive the image file and determine on basis of the image file a chemical and/or physical property, such as a pH value, of the one or more samples in the container 214.

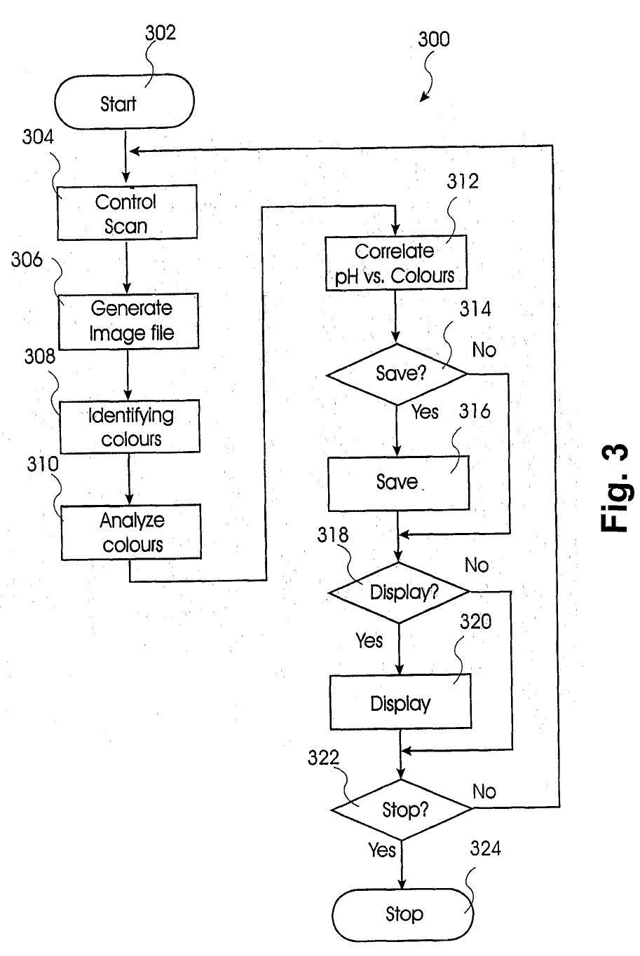

FIG. 3, shows a computer program designated in entirety by reference numeral 300. It is to be understood that the computer program 300 is according to a preferred embodiment of the present invention and executed by the system and/or method described with reference to FIG. 1 and FIG. 2.

The computer program 300 may be written in any high or low level computer language and may be compiled by any compiler known to a person skilled in the art.

The computer program 300 comprises a start procedure 302 for initialising internal communication between processor or processors and memory unit and external communication between the computer running the computer program 300 and the peripherals associated with the computer. Peripherals may comprise display unit, flat bed scanner, digital camera, mouse, joy-stick, hard disks, keyboards, robotic arms, relays, or any combination thereof.

The computer program 300 further comprises a control scan procedure 304 during which the computer program 300 controls a flat bed scanner and requests the flat bed scanner to perform a scan of a sample.

Furthermore, the computer program 300 comprises a generate image file procedure 306 during which an image file is generated on the basis of a scan of the sample. The image file may as described with reference to FIG. 1 and FIG. 2 comprise any format known to a person skilled in the art.

The computer program 300 identifies the colors of the sample during an identifying colors procedure 308. The colors indicate a specific pH level of the sample. The colors are analysed during an analyze colors procedure 310 during which image parameters, such as lightness chroma, Hue, saturation, angle (illuminating angle, viewing angle, aspecular angle), or any combination thereof, are determined. During a translation procedure 312 the image parameters determined during the analyse procedure 310 are correlated with a pH value.

The computer program 300 comprises a saving procedures 314, 318 and display procedures 318, 320, which may be implemented in a wide variety of ways. For example, by responding to operator action or by introducing dependencies such as flags identifying saving requests or displaying requests. Similarly, the computer program 300 comprises stopping procedure 322, 324, during which the computer executing the computer program is notified of termination. The stopping of the computer program 300 may be established by an operator's interaction or by flagging means.

The term flagging means should in this context be construed as a parameter or flag, which may be raised by any of the procedures of the computer program 300, or may in fact be a parameter or flag, which is raised by peripheral equipment or internal interrupt requests in the computer.

The method and system of the present invention can be used with the implementation of automated liquid-handling solutions, high-throughput (multichannel) manual pipetting techniques, medium-throughput sample filtration techniques, and computer automated customer report generation. Advantages of the invention include: increased sample turnover, and increased utility of the resultant data.

### EXPERIMENTAL SECTION

### Example 1

### The Color of Viscosity—a Method for Estimation of Texture

The texturing capabilities of Streptococcus thermophilus (STs) are very important in yogurt applications. Until now no fast method for screening bacterial cultures for their capability to add texture to a milk based matrix (e.g. yogurt) have been available. Currently the texture generated by a culture growing in milk is analyzed by simple ropiness scoring (a visual scoring) or by measurement of viscosity on a rheometer. Both methods are quite laborious and it is only possible to analyze a small number of samples. These methods are therefore not suitable for screening a large number of strains as in e.g. screening campaigns. Here we describe a new method for easy and fast estimation of the texturing capabilities/the viscosity of bacterial cultures growing in milk. The screening is performed in microtiter plates and by using robotics and therefore has a high throughput

The cultures are inoculated and acidified over night. Each well of the microtiter plate then corresponds to a model yogurt. The viscosity is estimated by the “The color of viscosity” method of the present invention based on diffusion of a color (Ruthenium Red) in a non-transparent matrix (e.g. milk). The principle of the method is that the thicker the matrix (the more viscosity) the longer time it takes for the color to diffuse through it. The color (Ruthenium Red) is applied to the surface of the model yogurt surface in the microtiter plate and the color (the hue angle) at the bottom of the well is recorded by a picture capturing device. In the present example a commercially available flat bed scanner is used for picture capturing. FIG. 2 describes the system in principle. In this example a HP ScanJet 6300 C with disabled color correction and disabled light correction was used for picture capturing.

When Ruthenium Red is applied to the surface of the matrix in the microtiter plate well the color at the bottom is still milky-white (hue angle ˜80°). When the diffusion of the color is completed, the color at bottom of the wells will be read as pink (as Ruthenium Red, hue angle ˜340), FIG. 3. The distance (angle) from full diffusion (completely pink at the bottom of the wells) is defined as DHue°, where a DHue° value of approximately 100° corresponds to no diffusion and a value of ˜0° corresponds to full diffusion. The DHue° is calculated as: 80° (determined hue angle of milk)−Hue° (360°−T2), if T2>340° (determined hue angle of milk+Ruthenium Red). If T2<340°; DHue°=80°.

### Materials and Methods

Strains (Table 1, below) were inoculated from a so-called Frozen Direct Vat Set culture (F-DVS) into M17 broth supplemented with 0.5 w/v lactose and incubated anaerobically over night at 37° C. A 2% v/v pre-dilution of the over night cultures were prepared manually in 5 ml Reconstituted Skimmed Milk. From this pre-dilution 100 ml was transferred into a microtiter plate (96 wells, Nunc) containing 0.2 ml of milk pr well, resulting in a final dilution for acidification of 1 v/v % of the over night culture. This inoculation was performed by a liquid handling robot and made in triplicates. The incubation was performed in an anaerobic incubator (Galaxy RS incubator). After incubation for 21 hours 5 ml of Ruthenium Red (5 mg/ml in water) was added by a liquid handling robot to the surface of each well. The plates (hue° at the bottom) were scanned each 3'rd minute at room temperature. The time for diffusion (Tdiff) was calculated.

### Calculations

The DHue° is defined as the distance (angle) from full diffusion. T1 Indicate the time for application of the color (Ruthenium red) at the surface of the matrix. At start the hue angle read at the bottom is ˜80° corresponding to milky-white. T2 is the time for full diffusion of Ruthenium Red. The hue angle read at the bottom corresponds to ˜340° (pink).

The calculated DHue° values was plotted versus time for different Streptococcus thermophilus strains. When the DHue°=0 it corresponds to full diffusion of Ruthenium red this value is the time for diffusion=Tdiff. Tdiff gives an estimation of the viscosity/texture of the matrix. A high Tdiff corresponds to a thick matrix which would indicate that the culture growing in the model yogurt gives rise to high viscosity/texture.

TABLE 1
Strains/cultures used in this example

| Identifier | Description |
|---|---|
| ST44 | Streptococcus thermophilus |
| ST4239 | Streptococcus thermophilus |
| ST124 | Streptococcus thermophilus |
| STFE2 | Streptococcus thermophilus |
| ST102 | Streptococcus thermophilus |
| ST10255 | Streptococcus thermophilus |
| ST4895 | Streptococcus thermophilus |
| TH4 | Streptococcus thermophilus |
| ST143 | Streptococcus thermophilus |
| CH1 | Mixed-yogurt culture |
| YC380 | Mixed - yogurt culture |
| YCX11 | Mixed - yogurt culture |
| YCX16 | Mixed - yogurt culture |
| YC180 | Mixed - yogurt culture |

### Results

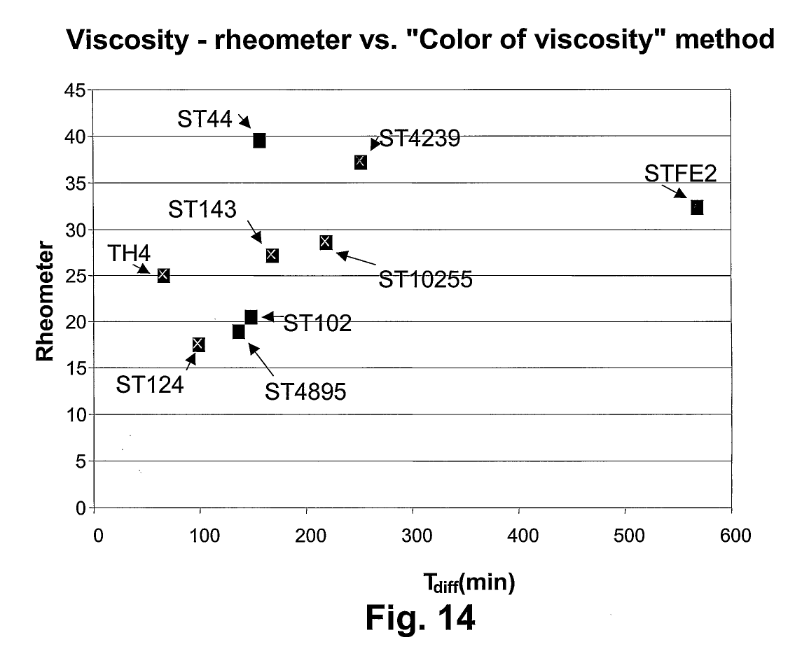

The Tdiff values for nine different Streptococcus thermophilus were plotted against their viscosity (FIG. 14) measured in a rheometer (Department of Fermented Milk, CH-Hoersholm) according to their standard protocol for measuring viscosity. As seen in FIG. 14 there is a good correlation between the two methods for most of the cultures. However, the cultures TH4 and ST44 results in a lower viscosity in “Color of viscosity” method compared to when measured in the rheometer. This can be explained by the fact that strain TH4 and ST44 is a fast and a slow acidifier, respectively, and the pH in the MTP was lower and higher, respectively, then the pH of the remaining control strains when initiating “The color of viscosity” assay. A too low pH may make the color escape the matrix due to syneresis resulting in a to low Tdiff determination. If acidification is too slow the result is higher pH that give less production of EPS and hereby less texture resulting in a fast diffusion (=to low Tdiff) in “The color of diffusion” assay.

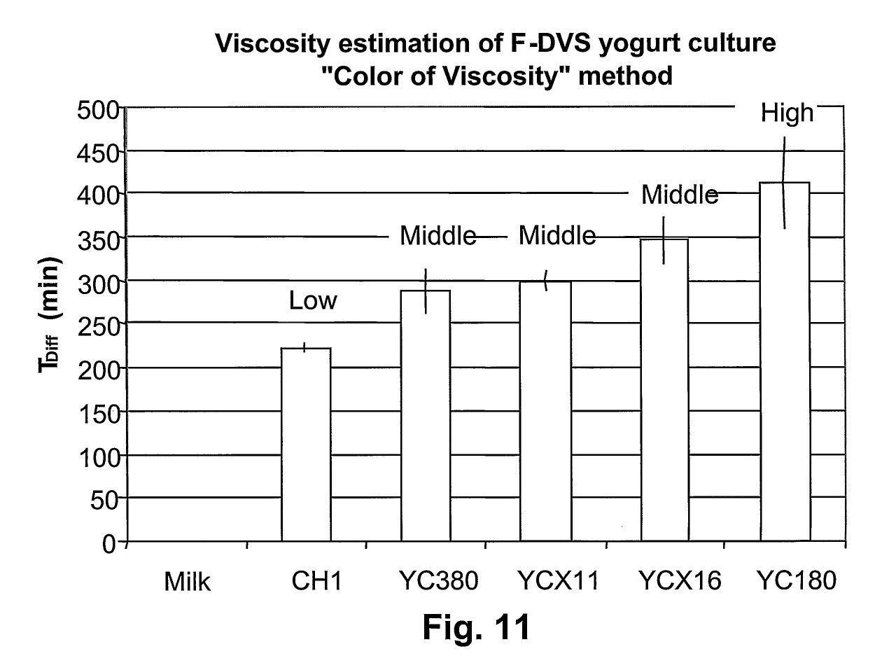

Further, by using “The color of viscosity” assay five commercial mixed yogurt cultures was tested. As seen in FIG. 11, it was possible to group the five tested yogurt cultures into groups of low, middle and highly texturing cultures based on the Tdiff determination. This grouping corresponds to the all ready know texturing capabilities of the commercial cultures.

### Conclusion

The “Color of viscosity” assay has been tested with 9 Streptococcus thermophilus cultures (FIG. 14) covering a wide range texturing cultures. As seen in FIG. 14 there is a good correlation between values obtained with a rheometer and by “The color of viscosity” assay.

The “Color of viscosity” assay has furthermore been tested with 5 cultures (FIG. 11) covering a range of low, middle and highly texturing cultures. By determination of the diffusion time of Ruthenium Red through the cultures after acidifications in microtiter plates, it was possible to distinguish low from highly texturing cultures. This grouping corresponds to the all ready know texturing capabilities of the commercial cultures.

The data presented here shows that the “Color of viscosity” assay has a promising potential as a fast and easy screening tool for isolation of cultures with desired texturing/viscosity properties.

### Example 2

### A System for Determining pH in Milk in Microtitre Plates

In this example the method of the present invention is used for measuring pH in a turbid medium (milk). The pH-values were obtained from color determinations made from a picture obtained by scanning of the microtitre plate. The results demonstrate surprisingly good accuracy (average −0.05 pH) and precision.

The colorimetric calculations in the present example is partly based on the publication: Minolta: Precise Color Communication, “color control from feeling to instrumentation” Minolta CO., Ltd, 1994, which is incorporated herein by reference.

### Materials and Methods

### Reconstituted Skimmed Milk

### Glucono Delta Lactone (GDL), Sigma Inc.

Lactococcus lactis (O-culture), R 604 (Chr. Hansen catalogue no.: 200113)
Ampicillin: 100 mg/ml MilliQWater (MQW)

### Indicators:

Bromcresol purple (Sigma Inc.): 50 mg in 1 ml 0.02N NaOH plus MQW to 25 ml
Bromcresol green (Sigma Inc.): 50 mg in 1 ml 0.02N NaOH plus MQW to 25 ml
Indicator milk: 2.5 ml of each indicator was added to 50 ml milk and mixed.

### Standards

To 20 ml indicator milk was added 450 mg GDL and left on the table overnight.

A range of standards (total 11) were prepared by mixing this solution with indicator milk in different ratios as appears from Table 2 below:

TABLE 2

| | |
|---|---|
| GDL milk = | 450 mg GDL in 20 ml milk |
| | 22.5 mg GDL/ml milk |

| ml indicator milk | ml GDL milk | mg GDL/ml milk | pH |
|---|---|---|---|
| 2.00 | 0.00 | 0.00 | 6.88 |
| 2.00 | 0.05 | 0.55 | 6.72 |
| 2.00 | 0.10 | 1.07 | 6.53 |
| 2.00 | 0.15 | 1.57 | 6.37 |
| 2.00 | 0.30 | 2.93 | 6.07 |
| 2.00 | 0.50 | 4.50 | 5.78 |
| 2.00 | 0.70 | 5.83 | 5.60 |
| 2.00 | 1.00 | 7.50 | 5.41 |
| 1.50 | 1.50 | 11.25 | 5.17 |
| 1.50 | 2.00 | 12.86 | 4.99 |
| 1.00 | 2.00 | 15.00 | 4.52 |

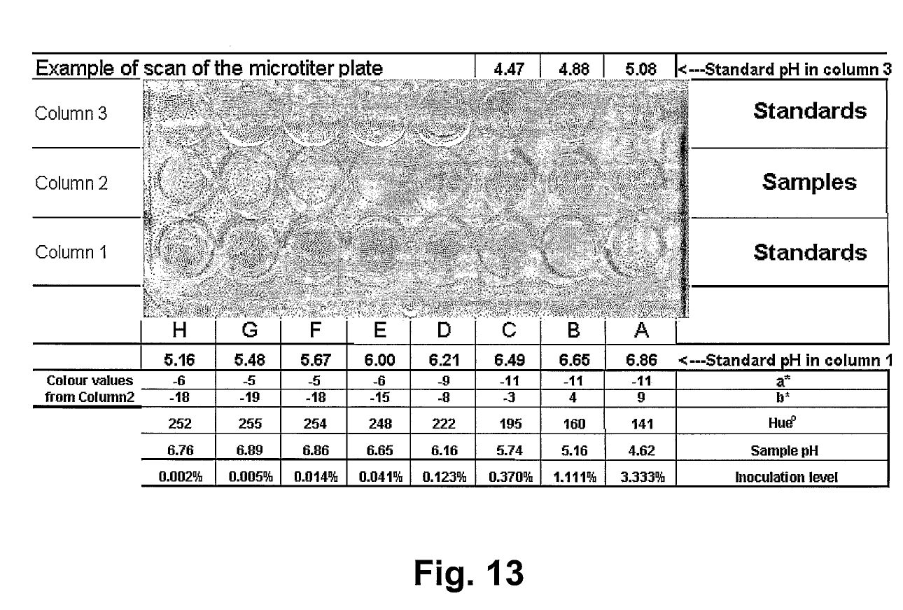

The standards were pipetted (200 μl) into the microtiter plate column 1 and column 3. FIG. 13 shows part of a flatbed scanned picture of the microtitre plate. Column 1 and 3 contained the pH standards. Column 3, Position D-H are empty wells. Column 2 contains samples with a decreasing concentration of culture (concentration of culture increasing from left to right). The inoculation level is stated in the line “Inoculation”, inoculation level is given in % vol/vol. The sample pH is calculated and the result of the calculation is showed in the line “Sample”.

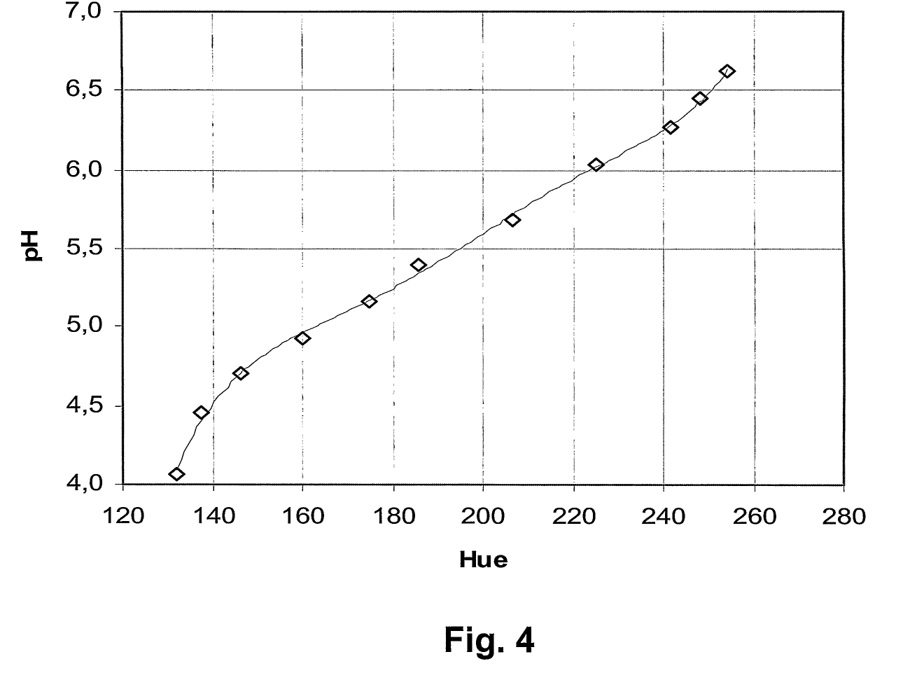

The standard curve is shown in FIG. 4.

### Samples

In column 2 was pipetted 200 μl indicator milk. 100 μl of a 10× dilution of the culture was pipetted into the first well (A2), sucked back and forth 10 times. 100 μl of this mixture was transferred to well B2, sucked back and forth 10 times. 100 μl of this mixture was transferred to well C2 and so on, creating a range of 3 fold dilutions.

The microtiter plate was incubated at ambient temperature in the scanner and scanned with regular intervals (30-60 min) throughout the day.

### Color Analysis

HP ScanJet 6300 C with disabled color correction and disabled light correction.

### Adobe Photoshop 5.0

“True color” scans were saved as jpg files.
Color values (L, a*, b*) were manually recorded in the centre of each well, 5×5 pixels using the “eyedropper” in the Adobe Photoshop 5.0 program.

### pH Measurements

pH of Standards and Samples was measured in the microtitre plate.

The pH meter used was IQ-Scientific Instruments equipped with an ISFET electrode calibrated prior to use.

### Calculations

Hue° values were calculated from the recorded a* and b* values using the formula:

Hue°=tan⁻¹(b*/a*)

(Minolta: Precise Color Communication, “color control from feeling to instrumentation” Minolta CO., Ltd, 1994).

Expression in Microsoft EXCEL: ATAN2(b*;a*)*180/PI( )

### Standards

The software “TableCurve 2D”, Systat Software Inc. (SSI), Richmond, Calif., USA, was used to find a suitable function describing the sigmoid character of the pooled standard curve.

f(Hue°)=(a+bx^{0.5}+cx+dx^{1.5}+ex²+fx^{2.5})*100

where x=Hue° and the coefficients are listed in Table 3 below:

TABLE 3

| | | Parameters | Values |
|---|---|---|---|
| Eqn | y = a + bx\hat{ }(0.5) + | a | −7415.83 |
| | cx + dx\hat{ }(1.5) + | | |
| | ex\hat{ }2 + fx\hat{ }(2.5) | | |
| Eqn # | 6701 | b | 2682.09 |
| r2 | 0.986 | c | −387.252 |
| DF Adj r2 | 0.985 | d | 27.91458 |
| Fit Std Err | 0.094 | e | −1.00445 |
| | | f | 0.014434 |

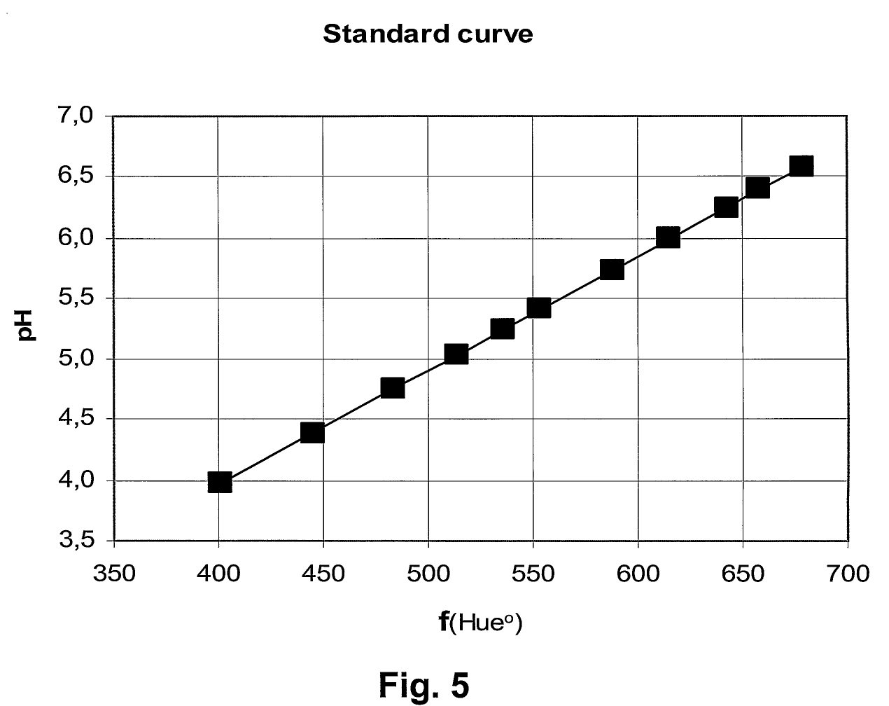

The standard curve of transformed (linearised) Hue value and pH is shown in FIG. 5.

### Results

Lactococcus lactis (O-culture) By using the regression result from the linear relationship between transformed color values and pH, the transformed color values from the samples were converted to pH.

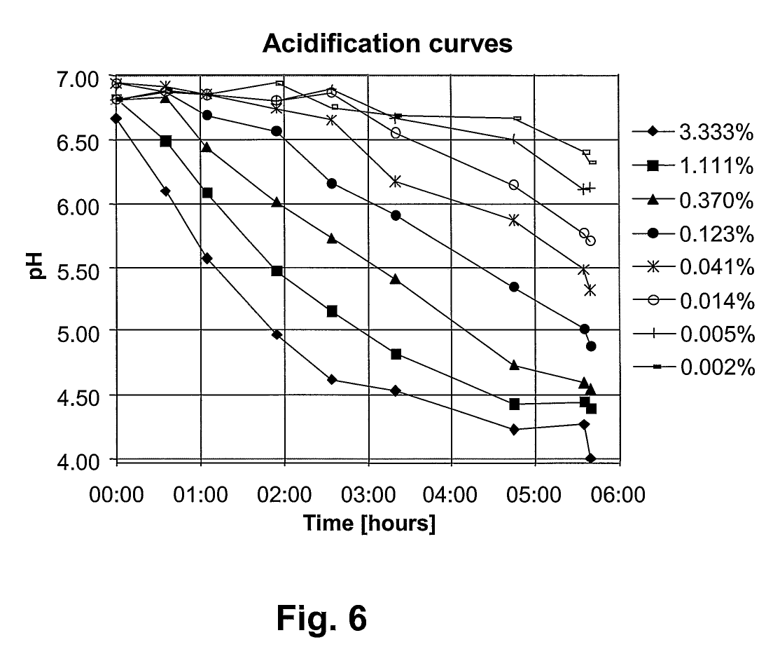

The acidification curves are shown in FIG. 6, wherein the legend indicates the inoculation level for eight different concentrations of a R 604 Lactococcus lactis (O-culture). The obtained curves correlates with similar acidification curves obtained by traditional techniques.

### Accuracy

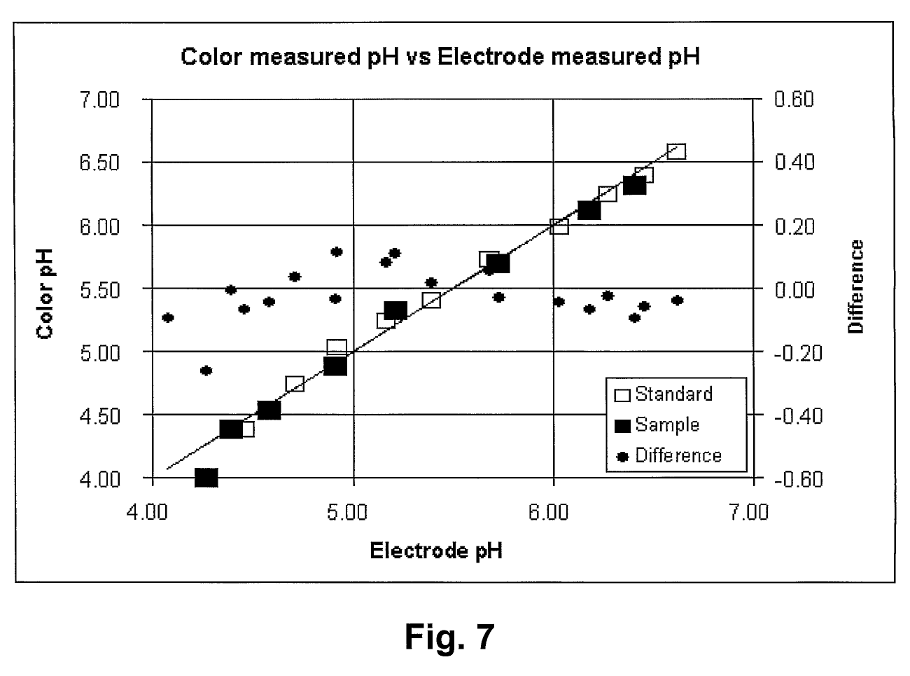

The accuracy of the pH determination for the Samples, defined as the average distance (absolute value) to the true value (electrode-pH), is 0.08 pH units. The average of differences is determined to −0.05. The accuracy for the last sampling point has been used in order not to destroy the gel and thereby create holes or alike interfering with the color measurements. The accuracy is demonstrated in FIG. 7, and will fulfil many demands. FIG. 7 shows the calculated pH (pH) versus pH measured by electrode in selected standards and samples after the incubation.

### Precision

If the two last sampling points are regarded as double determinations, the precision can be estimated to 0.085 pH unit (average of Standards and Samples). This is a conservative estimate since it is the lower part (<pH 5) of the standard curve which has the greatest uncertainty. The acidification curves obtained for the Lactococcus lactis (O-culture) are comparable with acidification curves obtained in 100× volumes (200 ml) (not shown). The precision is good for many applications. If needed, precision can be increased to get more differentiated results (decimals/more digits) for the color measurements.

### Conclusions

This study shows that it is possible to determine pH in milk in microtitre plates by measuring the color of the milk. Accurate determinations are possible and precision is within acceptable limits for a range of applications. One of the problems using small volumes (here 200 μl) is how to inoculate, handling of dilutions etc. The 96 wells plate used in this study provide an acceptable solution in practice. However, it is contemplated that the method can be extended to 384 well microtiter plates by covering the plate with at suitable seal ensuring no evaporation of the test medium. 4 plates can be scanned simultaneously giving 384 pH “readings” by the click of the mouse (using 96 wells microtitre plates). With the accuracy and precision described here, the method is applicable for screening studies. Improvements of accuracy and precision will open up for extended use. Scanners are cheap devices used in all offices and software requirements are small.

### Example 3

### Assay for the Determination of Phage Infection in Lactic Acid Bacteria

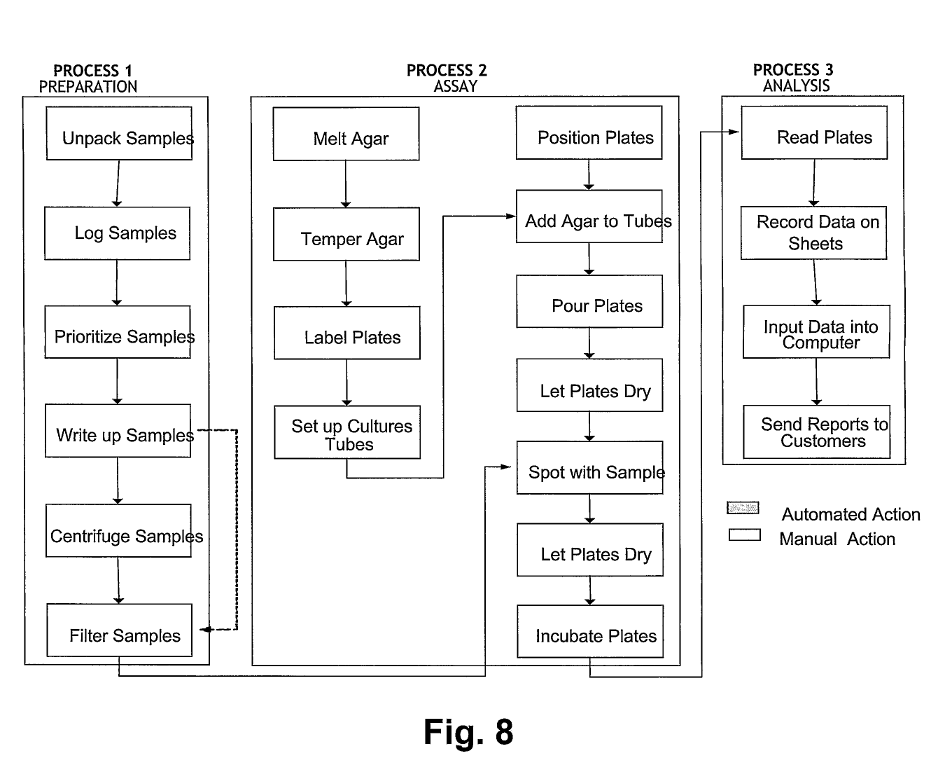

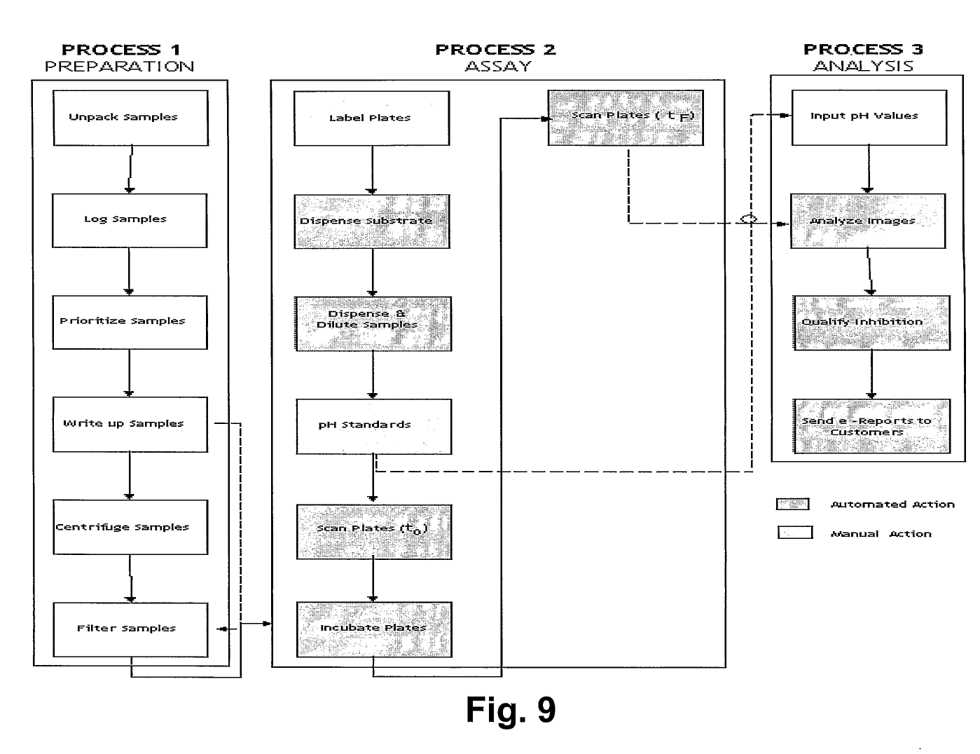

Many lactic acid bacteria that are used in the fermentation of foodstuffs are highly susceptible to attack by bacteriophages (phages). Loss of starter culture activity as a result of phage attack continues to be regarded as one of the most costly and persistent industrial problems. In recent years, the lactic acid bacteria (LAB) are being further exploited for the manufacture of industrial chemicals (e.g. lactate) and employed as vehicles for the delivery of biologics (e.g. vaccines and enzymes). With the expansion of fermentation and bioprocessing systems reliant on LAB, disruption by bacteriophage remains a growing concern. We describe herein a high-throughput assay that indirectly measures culture acidification over time by directly measuring the color of the growth substrate, which is affected through the activity of pH-responsive indicator dyes. A comparison between the various procedural steps of the calorimetric microassay of the invention and the current spot test for testing whey samples for bacteriophage infection can be made by comparing the flow diagrams of FIG. 9 with the flow diagrams of FIG. 8. As can be seen from FIG. 8 none of the required actions can be automated whereas about one third of the actions of the microassay of the invention can be automated.

### Materials and Methods

### Reconstituted Skimmed Milk

pH-meter
HP Scanjet 4670 Scanner with disabled color and light correction
A mesophilic Lactococcus lactis culture sensitive to lactococcus phage c2

### Incubators at 30° C.

Bromcresol purple (Sigma Inc.) (BCP): 50 mg in 1 ml 0.02N NaOH plus MQW to 25 ml
Bromcresol green (Sigma Inc.) (BCG): 50 mg in 1 ml 0.02N NaOH plus MQW to 25 ml

### Glucono Delta Lactone (GDL), Sigma Inc.

Indicator milk (IM): 2.5 ml of each indicator was added to 50 ml milk and mixed Glucono-delta-Lactone (GDL) milk (GDLM): GDL added to IM at 10% (w/v).

### Standards

12 colorimetric pH standards were prepared by combining GDL milk with 1M as described previously.

### Samples

As appropriate, a 96 well microtiter plates was used to house the experiment.

In position A1 to B12 are duplicate pH standards, used to establish the pH determination of the assay. Position C1 contained, Indicator Milk+10⁸ PFU/ml phage and no indicator bacteria; C2, Indicator Milk+10⁸ PFU/ml phage+indicator bacteria; C3, Indicator Milk+10⁶ PFU/ml phage+indicator bacteria; C4, Indicator Milk+10⁴ PFU/ml phage+indicator bacteria; C5, Indicator Milk+10² PFU/ml phage+indicator bacteria; C6, Indicator Milk and indicator bacteria only. Position D1 to D6 is a duplicate of C1 to C6.

Once the growth substrate, hosts, and phages were added to the microtitre plates, they were incubated at approximately 30° C. and periodically scanned (approximately once every hour). The data generated for individual host arrays was analyzed when the control wells (IM−phage+host) reached a target pH of approximately 5.5 (which was determined by comparison to the pH standard). The change (in pH (pH) between the control (IM−phage+host) and the test (IM+phage+host) wells was determined as a function of color change (color), as described in Example 2.

### Results

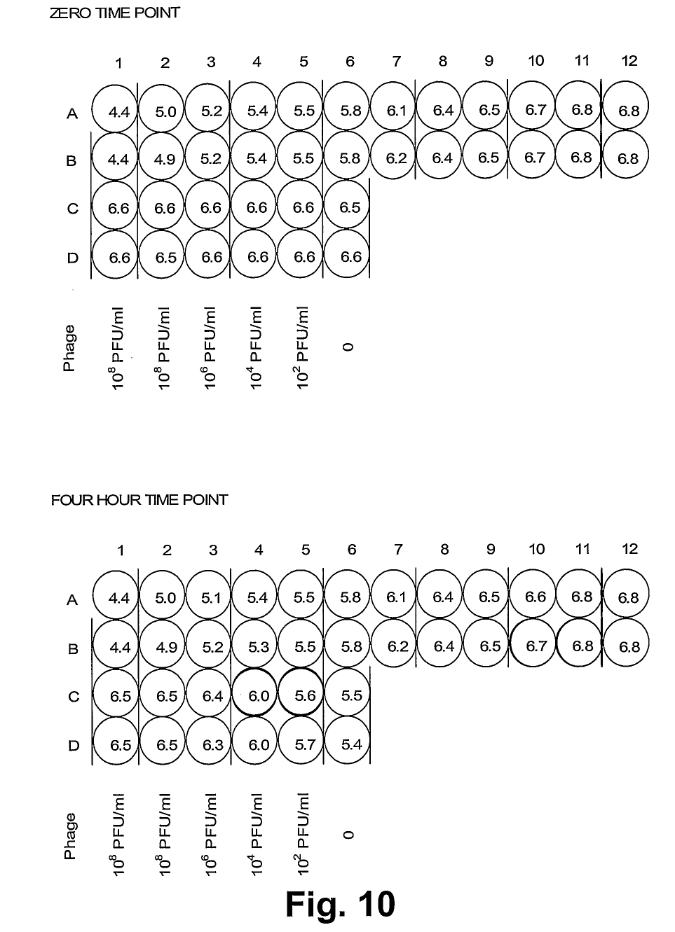

The result of one experiment is shown in FIG. 10.

In the absence of phages, the host strain acidified the medium to the target pH of 5.5 after 4 hours. In the presence of high levels of phages (e.g. 10⁸ PFU/ml), the host bacteria were quickly lysed in all cases. As a result, the bacteria were unable to reduce the pH of the growth substrate over the course of the assay. The method was sensitive enough to detect even very low levels of phages present in the medium (e.g. 10² PFU/ml), and could differentiate between intermediate levels of phage (e.g. 10⁶ to 10⁴ PFU/ml).

### Conclusions

This example illustrates that the method and system for calorimetric determination can be applied to assay for the presence of bacteriophages.

### Example 4

### Precision of the pH Measurements in Milk

To quantify the precision of the pH measurements in milk the following experiment was performed.

Indicator milk was added to a Flat bottom 96-wells microtiter plate (NUNC), (Indicator milk: 50 ml Reconstituted Skimmed Milk added 2.5 ml of each indicator, Bromcresol purple and Bromcresol green, as described in Example 1). The standards were pipetted (200 μl) into the wells of the microtiter plate and scanned. The plate was placed on Canoscan 5000 (Canon Inc.) scanner.

After temperature stabilization (60 min) the microtiter plate was scanned every 2 min. pH was calculated from the obtained hue values using a costumer developed computer program pH-Scan 2.0.00, provided exclusively to Chr. Hansen A/S by HNH Consult Aps, Stoevring, Denmark. The pH-Scan 2.0.00 program automatically performs all functions performed by computer programs described in Example 2 and outlined in FIGS. 2 and 3.

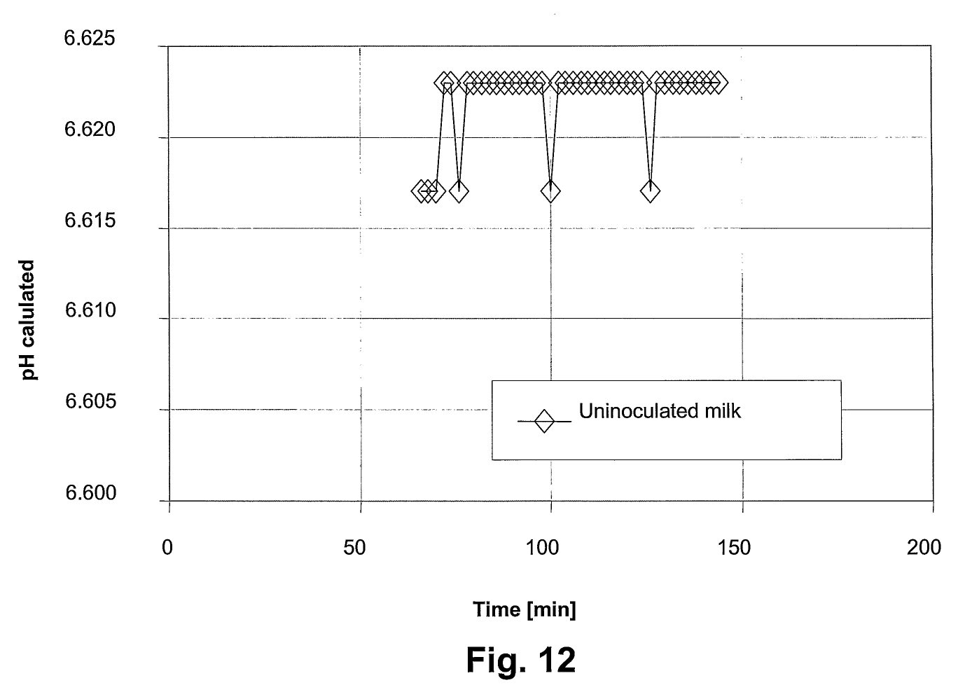

The observed results are shown in FIG. 12.

From the 40 individual measurements shown in FIG. 12 the average pH value and standard deviation were calculated to average=6.6221 (pH units); 1×S.D.=0.0022 (pH units). This is an surprisingly high precision which are comparable to the precision obtained with high precision pH meters.

1. A method for simultaneous determination of a biological, chemical and/or physical property of a plurality of individual samples of a turbid medium comprising the steps:
i) arranging said individual samples of the turbid medium comprising a color indicator in an array;
ii) allowing said color indicator to interact with said samples;
iii) providing picture capturing means for capturing a digital image of the color developed on a surface of said samples following said interaction;
iv) using said digital image to obtain a digital value representation for said property, said value representation being used for calculating a value for said property.

2. A method of claim 1 further comprising the step of comparing said digital value with the values obtained from a standardised set of samples having a range of known values representing said property to obtain a calculated value for said property.

3. A method according to claim 1, wherein said array is in the form of a container, having a plurality of wells arranged in an array format.

4. A method according to claim 1, wherein said container has at least a partly transparent surface.

5. A method according to claim 1, wherein said picture capturing means for determination of color is a color-enabled photoelectric scanning device, that produces a digital color representation of said surface.

6. A method according to claim 1, wherein said picture capturing means is a scanning device.

7. A method according to claim 1, wherein said scanning device is one of a line-by-line operating autofeed scanner, a flatbed scanner, a digital video camera or a digital camera.

8. A method according to claim 1, wherein said scanning device operates on an at least partly open or transparent end of said container to generate an image file recording of the color of said sample.

9. A method according to claim 8, wherein said scanning device operates through the bottom of said container.

10. A method according to claim 1, wherein digital image processing methods are used to obtain an image file for determining the measuring positions from the digital color representation of said surface and for calculating said value for said property.

11. A method according to claim 1, further comprising the step of illuminating the at least partly transparent surface of the container in connection with determining said color.

12. A method according to claim 8, further comprising the steps:
i) analyzing said image file and generation of data values for image parameters by means of an analyzer; and
ii) translating said data values for image parameters to a value representing said biological, chemical and/or physical property of said sample by means of said analyzer.

13. A method according to claim 1, wherein said medium is liquid, semi-liquid or a gel.

14. A method according to claim 1, wherein said medium is selected from the group consisting of biological fluids, emulsions, latex emulsions, mayonnaise, salad dressings, skin lotions and skin tonics.

15. A method according to claim 1 wherein said medium contains live microorganisms, yeasts, lactic acid bacteria or yeasts and lactic acid bacteria.

16. A method according to claim 8, wherein said image file comprises an image format.

17. A method according to claim 12, wherein said image parameters comprise lightness, chroma, Hue angle, or any combination thereof.

18. A method according to claim 17, wherein said Hue angle determined from said image file is correlated with a pH level of said sample.

19. A method according to claim 5, wherein said scanning device scans said container, said scanning comprises scanning at predetermined time intervals.

20. A method according to claim 12, wherein said analyzing of said image file is performed in predetermined regions of said container.

21. A method according to claim 12 which further comprises saving said image parameters in a data file.

22. A method according to claim 21, wherein said data file is a comma-separated-value type file, a space-separated-value type file, a text type file, or any combinations thereof.

23. A method according to claim 21 further comprising presenting said data file in graphical or textual form by means of a display.

24. A method according to claim 12, wherein said analyzer comprises a digital processor.

25. A method according to claim 24, wherein said analyzer further comprises a memory device for storing an analyzing program code to be executed by the processor, for storing image files recorded by the scanner, and for storing data values generated by the analyzer.

26. A method according to claim 25, wherein said memory device and/or said scanning device is adapted to connect to said processor through a computer network.

27. A method according to claim 21, wherein said analyzer further comprises a display for displaying progress of said analyzing of said image file and/or displaying said data file.

28. A method according to claim 1, wherein said property is the pH of the medium.

29. A method according to claim 1, wherein said color indicator is a pH indicator.

30. A method according to claim 29, wherein said pH indicator is adapted to indicate pH levels between 3 and 8, the pH indicator being selected from the group consisting of bromocresol green, bromocresol purple, bromophenol blue, bromothymol blue, chlorophenol red, phenol red.

31. A method according to claim 1, wherein said medium is milk, a processed milk product, whey, a bacteriological medium or a combination thereof.

32. A method according to claim 1 or 3 which further comprises adding bacterial cultures to said sample in said array or container.

33. A method according to claim 31, wherein determination of pH is an indication of bacteriophage infection of the lactic acid bacteria.

34. A method according to claim 1 or 3, wherein said property is the viscosity of the medium.

35. A method according to claim 34, wherein said medium is yogurt.

36. A method according to claim 35 wherein said color indicator is brilliant blue or Ruthenium Red.

37. A method according to claim 34, wherein said color indicator is added to the top surface of or into the sample after adding the sample to the array or the container.

38. A method according to claim 36, wherein said color indicator is allowed to interact with said sample according to a predetermined time wherein a development at the bottom of said sample surface is known to correspond to a specific viscosity of said medium.

39. A system for determining a chemical or physical property of a turbid medium comprising:
i) a container for containing a sample of said medium;
ii) an indicator to be introduced in said sample, said indicator being adapted to indicate a specific value of said property by a specific color;
iii) an incubator for supporting said container and incubating said sample contained in said container;
iv) a scanner or digital camera for scanning said container, thereby generating an image file recording the color of said sample having reacted with the indicator,
v) an analyzer for analyzing said image file and generating data values for image parameters for said image file and determining the value of said property of said sample from said image parameters.

40. A system of claim 39 wherein said property is the acidity of said medium as measured by pH and said indicator is a pH indicator.

41. A system of claim 39 wherein said property is the viscosity of said medium and said indicator is a colored substance characterised in having a time for penetration of said sample which is correllated with the viscosity of said sample.

42. (canceled)

43. A method according to claim 1, wherein the container is a microtitre plate having between 2 and 4000, 6, 24, 96, 384 or 1536 wells.

44. A method according to claim 3, wherein said container has transparent top or bottom.

45. A method according to claim 1, wherein said medium is selected from the group consisting of dairy products, oil products, fruit juice products, jelly, spice products, beverages, whole blood, serum, or any combination thereof.

46. A method according to claim 1, wherein said medium contains yeasts or lactic acid bacteria or both.

47. A method according to claim 8, wherein said image format comprises Synchronized Multimedia Integration Language (SMIL) format, any JPEG format, any Graphics Interchange Format (GIF), Computer Graphics Metafile, TIFF, BIFF, bmp, Clear, FITS, NFF, OFF, PCX, PNG, TGA, XBM, mod, Portable Document Format (PDF), Portable Network Graphics, Portable Pixmap, progressive coding, Quicktime, RIFF, Self Extracting Archive, sequential coding, server-parsed HTML, sprite, Tagged Image File Format, targa, Targa Graphics Adaptor, thumbnail, wav, WebCGM, wireless bitmap, xpm or a different frame rate video or similar format.

48. A method according to claim 5, wherein said predetermined time intervals comprise the intervals of about 0.00001 second to about 60 minutes, or about 0.00001 second to about 120 minutes.

49. A method according to claim 24, wherein said digital processor is included in a computer, a server system, a personal digital assistant, a cell phone, or any combination thereof.

50. A method according to claim 25, wherein said memory device and/or said scanning device is adapted to connect to said processor through a dedicated line network, a local area network, a wide area network, a metropolitan area network, or an inter-network.

51. A method according to claim 27, wherein said display includes a monitor for presenting textual or graphical data, a personal computer monitor, a personal digital assistant monitor, a cell phone display, or any combination thereof.

52. A method according to claim 31, wherein said medium comprises live lactic acid bacteria.
